(19) United States
(12) Patent Application Publication
(10) Pub. No.: US 2004/0215125 A1
(43) Pub. Date: Oct. 28, 2004

(54) Bifurcated graft for dialysis

(76) Inventor: Donald W. Brown, Bowling Green, KY (US)

Correspondence Address: Donald W. Meeker, Patent Agent, 924 East Ocean Front #E, Newport Beach, CA, 92661, US

(21) Appl. No.: 10/424,496

(22) Filed: Apr. 27, 2003

(51) Int. Cl. A61F 2/06

(52) U.S. Cl. 604/6.16; 623/1.35; 604/8

(57) ABSTRACT

A tubular, bifurcated Y-shaped prosthetic graft is provided for use between the artery and the vein of a patient undergoing dialysis treatment. The leg of the Y in the artery and the two arms in the vein insure a through flow of blood into the vein, so scarring in the vein does not prevent blood flow. The ends of the graft are capable of attachment in any manner, such as being sewn to the artery and vein. A sealant may be used to prevent leakage at the attaching points. The graft is made of PTFE or Dacron. The lengths and diameter of each limb of the Y vary depending on the size of the patient and the location of attachment.

### BACKGROUND OF THE INVENTION

1. Field of the Invention

The present invention relates to prosthetic vascular grafts and, more particularly, to a bifurcated vascular access graft adapted for use in the dialysis field.

2. Description of the Prior Art

Dialysis treatment of individuals suffering from renal failure requires that the blood be withdrawn and cycled through a dialysis machine that performs the function of the failed kidneys. This process, termed hemodialysis, must be repeated regularly and thus requires repeated puncture wounds using a dialysis needle. Moreover, dialysis requires a relatively rapid blood flow rate, typically above 200 ml/min, and so the dialysis needle is relatively large. Host vessels have insufficient strength to withstand collapse from such frequent puncturing with large bore needles.

A common technique to provide vascular access for hemodialysis, therefore, is to connect a prosthetic arteriovenous (AV) graft or shunt between an artery and a vein in, for example, the arm. The AV graft is constructed to withstand numerous puncture wounds or “sticks” without collapse.

Conventional AV grafts are typically constructed of woven or knitted Dacron or gortex (PTFE). Conventional AV grafts must be implanted for at least two weeks prior to puncture so that an intimal layer of fibrotic tissue has an opportunity to attach to the luminal surface of the graft. The layer of fibrotic tissue prevents blood leakage through the wall of the graft upon puncture. Prior to the time at which the graft can be safely punctured without leakage, a central venous catheter (CVC) must be utilized to collect the blood required for cycling through the dialysis machine. The CVC is needed because of the relatively high blood flow rates involved. For certain patients, however, use of a CVC is contraindicated.

Infection is always a problem in such invasions of the body. Another problem with AV graft is the end of the tube and the vein that it connects to forms a layer of cell hyperplasia that is similar to scar tissue that eventually leads to blockage. Prior art patents have not adequately addressed this problem.

Prior art U.S. Pat. No. 5,922,022, issued Jul. 13, 1999 to Nash, indicates a system and method of use for effecting the bypass or other anastomosis of a portion of a native blood vessel, duct, lumen or other tubular organ within the body of a living being. The system includes a connector assembly and a deployment instrument for carrying the device to the desired position within the vessel, duct, lumen or tubular organ. The system includes a piercer-dilator instrument to form an opening in the wall of the vessel, duct, lumen or tubular organ into which the connector assembly is deployed by the deployment instrument. The connector assembly is at least partially formed of a resorbable material and includes movable members for securing it to the tissue of the vessel, duct, lumen or tubular organ contiguous with the opening. Other components may be included in the device for expediting the anastomosis procedure, with or without the use of sutures. Moreover, the system can be used to bypass of at least two coronary arteries by a common connection with the aorta utilizing at least two bypass grafts, a single upstream anastomosis connector, a pair of downstream anastomosis connectors, and a common bifurcated midstream anastomosis connector, for securement between the aorta and the coronary arteries.

Prior art U.S. Pat. No. 6,102,884, issued Aug. 15, 2000 to Squitieri, is for a hemodialysis and vascular access system that comprises a subcutaneous composite PTFE silastic arteriovenous fistula that has an indwelling silastic venous end. The silastic venous end is inserted percutaneously into a vein and a PTFE arterial end which is anastomosed to an artery. Access to a blood stream within the system is gained by direct puncture of needle(s) into a needle receiving site having a tubular passage within a metal or plastic frame and a silicone upper surface through which needle(s) are inserted. In an alternate embodiment of the invention, percutaneous access to a blood stream may be gained by placing needles directly into the system (i.e. into the PTFE arterial end). The invention also proposes an additional embodiment having an arterialized indwelling venous catheter where blood flows from an artery through a tube and a port into an arterial reservoir and is returned to a vein via a port and a venous outlet tube distinct and distant from the area where the blood from the artery enters the arterial reservoir. The site where blood is returned to the vein is not directly fixed to the venous wall but is free floating within the vein. This system provides a hemodialysis and venous access graft which has superior longevity and performance, is easier to implant and is much more user friendly.

Prior art U.S. patent application Ser. No. 20020193872, published Dec. 19, 2002 by Trout, provides a surgical component and a surgical component assembly, such as a prosthetic graft, and methods of their use. In particular, embodiments of the present invention relate to a surgical component and surgical component assembly for use during a surgical procedure for repairing a vessel. The surgical component in accordance with the present invention may comprise a body portion having at least one orifice located thereon. The body portion and orifice(s) may be customized to an individual surgical patient and may further Prior art be marked by indicators for use during a surgical procedure.

Prior art U.S. Pat. No. 4,822,341, issued Apr. 18, 1989 to Colone, concerns a vascular access fistula that includes a smooth, continuous PTFE tube that has a hard sintered tube section integrally joined at its opposing ends with expanded PTFE tube sections. Inlet and outlet access port holes are formed in the hard sintered tube section to provide acute access to the fistula. A port collar surrounds the hard sintered tube section to facilitate the attachment of dual lumen tubing thereto. Following implantation of the access fistula, the dual lumen tubing temporarily extends through the skin to provide acute access while the access fistula heals. The port collar houses a slide valve that selectively couples the inlet and outlet access port holes to the dual lumen tubing for acute access. After the access fistula has healed, the slide valve is closed, the dual lumen tubing is cut off from the port collar, and chronic vascular access is provided by percutaneous cannulation of the implanted PTFE tube.

Prior art U.S. Pat. No. 5,741,325, issued Apr. 21, 1998 to Chaikof, is for a self-expanding intraluminal composite prosthesis that is comprised of a rigid reinforcing component and sealing component. The prosthesis may be fabricated as either a straight or bifurcated tubular structure and is applicable to the treatment of any bodily passage including, but not limited to, vascular applications, e.g., aneurysms, arteriovenous fistulas, as well as stenotic regions of the peripheral circulation which have been percutaneously dilated but are at high risk for restenosis. The major attributes of this prosthesis can include the use of a unique multilayered biaxial braid which thereby creates a homogeneously blended composite with isotropic deformation and expansion characteristics and an associated high contraction ratio. The use of multiple layers allows for the fabrication of a device of varied porosity while retaining adequate tensile or mechanical wall strength.

Prior art U.S. Pat. No. 6,485,513, issued Nov. 26, 2002 to Fan, indicates a prosthetic vessel graft assembly that has a tube of synthetic graft material surrounding a stent at its distal end, together with a plurality of collapsible anchors projecting radially from the stent. The assembly mounts on an introducer that may be tunneled through skin or inserted in an opening and pushed down to a vessel for introducing the graft through the vessel side wall. The introducer is configured to follow a guide wire that is inserted via a needle through an opening in the side wall of the vessel. A peel-away sheath covers the stent, graft, and anchor members at the distal end keeping them collapsed against the tubular body.

Prior art U.S. Pat. No. 6,261,257, issued Jul. 17, 2001 to Uflacker, puts forth a dialysis-access graft fistula having self-sealing ports for use in hemodialysis. The self-sealing ports can be repeatedly used for the cannulation required for the blood flows associated with hemodialysis. The ports self-seal after each puncture.

Prior art U.S. Pat. No. 6,319,279, issued Nov. 20, 2001 to Shannon, illustrates a vascular access graft that is radially supported and self-sealing upon puncture with, for example, a dialysis needle. The graft has at least one access segment that is formed by an inner layer, an intermediate layer, and outer layer. The intermediate layer has, in longitudinal cross-section, regions of different densities. Radial support members within the intermediate layer prevent collapse of vascular access graft and may be formed of a material that has a lower melting temperature than the other components of the graft. A porous or low-density material is provided between the radial support members to permit blood seepage therein, and the graft is formed by heating to cause the radial support members to melt slightly into the interstitial spaces of the low-density material. The radial support members may be individual turns of a helical coil of FEP, and the low-density material may be compressed PTFE “cotton”. The inner and outer layers may also be formed of PTFE. An adhesive layer of FEP may be provided closely surrounding the inner layer to further anchor the various components of the graft during the step of heating. The inner layer extends on either side of the access segment to provide junction segments that can be cleanly trimmed to size, and which can be used for graft cannulation in lieu of the access segment after suitable tissue ingrowth. A method of manufacture of the vascular access graft is also provided.

Prior art U.S. Pat. No. 5,910,168, issued Jun. 8, 1999 to Myers, shows a vascular graft in the form of a tube or flat sheet of biocompatible material having an outer covering of deflectably secured material such as porous film, fibers, discrete pieces of material, or combinations thereof. The vascular graft may be of tubular form for replacing or bypassing entire segments of veins or arteries or alternatively may be in the form of a flat sheet useful for repairing portions of the circumference of veins or arteries. The tube or flat sheet of biocompatible material and the outer covering of deflectably secured material are preferably of porous polytetrafluoroethylene. The vascular graft is useful for dialysis access in that it offers a reduction in blood leakage when the vascular graft is punctured by a dialysis needle and the needle is subsequently removed. It is also useful for reducing suture hole bleeding resulting from holes created through the graft by penetration of the graft with a suture needle.

Prior art U.S. Pat. No. 5,575,817 issued Nov. 19, 1996 to Martin, discloses an apparatus and method for reinforcing a bifurcating blood vessel. The apparatus comprises two sections that form an inverted Y-shape graft prosthesis when joined together inside the blood vessel. The method comprises inserting the two sections into the bifurcating blood vessel by encasing the sections in retractable membranes mounted on two catheters, inserting the catheters into the blood vessel, deploying and attaching the first section to the vessel, deploying the second section and joining it to the first section.

What is needed is a multiple tube connection to the vein so that the voluminous blood flow continues regardless of the scarring which normally blocks a single tube.

### SUMMARY OF THE INVENTION

An object of the present invention is to provide a bifurcated Y-shaped prosthetic graft between the artery and the vein in dialysis treatment with the leg of the Y in the artery and the two arms in the vein to insure a through flow of blood into the vein so that scarring in the vein does not prevent the blood flow.

Another object of the present invention is to provide a graft that will reduce the number of surgeries required because of clotting caused by hyperplasia (scar tissue) at the venous end.

In brief, the bifurcated graft of the present invention is fabricated in the shape of a Y and preferably made of PTFE or Dacron. It can be attached in any manner, such as being sewn to the artery and vein and also using a sealant to prevent leakage at the attaching points. The lengths of each limb of the Y can vary depending on the size of the patient and the location of attachment. The usual diameter is 6 mm but can be larger.

An advantage of the present invention is in providing a graft for use in kidney dialysis.

Another advantage of the present invention is providing two separate vein locations.

An additional advantage of the present invention is that it should decrease the number of surgeries required.

One more advantage of the present invention is in increasing patient satisfaction.

Yet another advantage of the present invention is in lowering the cost to our health care system.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a perspective view of the Y-shaped bifurcated vascular access graft attached between an artery and a vein;

FIG. 2 is a perspective view of the Y-shaped bifurcated vascular access graft slightly bent upwardly at each end to show the tube openings.

### BEST MODE FOR CARRYING OUT THE INVENTION

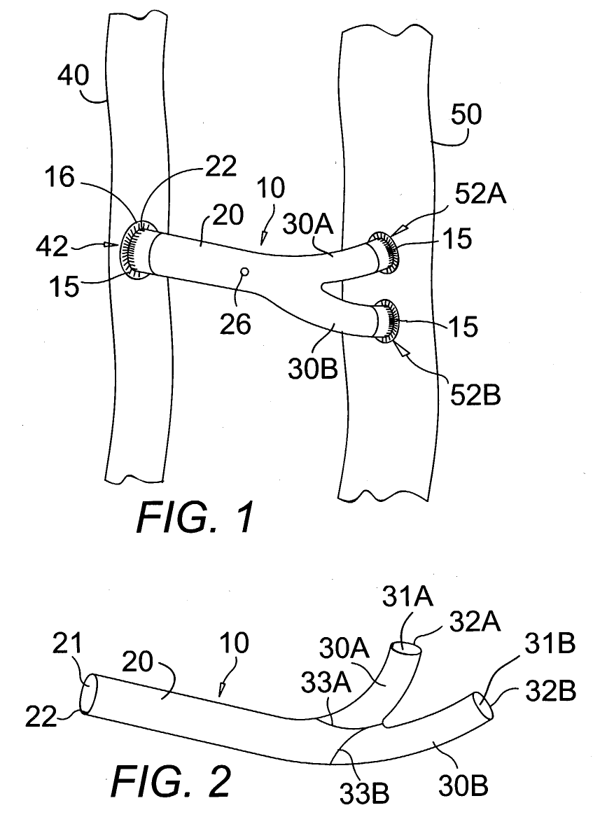

In FIGS. 1 and 2, a bifurcated vascular access graft is provided for use in kidney dialysis. The graft has a tubular configuration 10 that is a Y shape with the arterial tube 20 comprising the leg of the Y and the two venous tubes 30A and 30B comprising the arms of the Y. The arterial tube 20 has an interior opening 21 therethrough, as shown in FIG. 2. The arterial tube 20 has a distal end 22 configured for attachment to an artery 40 of a human in an arterial opening 42 so that the interior of the arterial tube 20 is capable of communicating with an interior of the artery 40.

The tubular configuration 10 also comprises at least two venous tubes 30A and 30B. Each of the two venous tubes 30A and 30B has a proximal end 33A and 33B interconnected with the arterial tube 20 and a distal end 32A and 32B capable of being attached to a vein 50 of the human. Each of the at least two venous tubes 30A and 30B are connected to the vein 50 at a different location in vein openings 52A and 52B. Each venous tube 30A and 30B has an interior opening 3 1A and 31 B therethrough with the venous tube openings 31A and 31B each communicating between the arterial tube interior opening 21 and an interior of the vein 50 of the human. This allows that blood from the artery 40 of the human is capable of flowing into the arterial tube 20 and out of the venous tubes 30A and 30B into the vein 50 of the human. The tubular 10 configuration of the graft is fabricated preferably of PTFE or Dacron. Each of the tubes 20, 30A and 30B of the graft has a diameter of at least 6mm.The distal end of the arterial tube 22 and the distal ends 32A and 32B of the venous tubes 30A and 30B are each capable of attachment to the artery 40 and the vein 50 respectively by a sewing means indicated by threads 15. Each of the distal ends 22, 32A and 32B is further capable of being sealed to the artery 40 and the vein 50 with a sealant 16.

A means is provided, such as a sealable opening 26 in the tubular configuration 10, for interconnecting a kidney dialysis machine (not shown), via a kidney dialysis tube, to the tubular configuration 10 so that the blood flows through the kidney dialysis machine prior to flowing into the vein 50 of the human.

In practice, a bifurcated graft with a tubular configuration 10 in a Y shape with the arterial tube 20 comprising the leg of the Y and the two venous tubes 30A and 30B comprising the arms of the Y, are affixed to the patient's artery 40 and vein 50 respectively. The distal end of the arterial tube 22 is attached to the artery opening 42 and the distal ends 32A and 32B of the venous tubes 30A and 30B are attached to the vein openings 52A and 52B by a sewing means indicated by threads 15. Each of the distal ends 22, 32A and 32B may then be sealed to the artery 40 and the vein 50 with a sealant 16. Following surgery a sealable opening 26 in the tubular configuration 10 may be accessed for interconnecting a kidney dialysis machine (not shown), via a kidney dialysis tube, to the tubular configuration 10. This allows the blood to flow through the kidney dialysis machine prior to flowing into the vein 50 of the human.

It is understood that the preceding description is given merely by way of illustration and not in limitation of the invention and that various modifications may be made thereto without departing from the spirit of the invention as claimed.

What is claimed is:

1. A bifurcated vascular access graft for use in kidney dialysis, the graft comprising:
a tubular configuration comprising an arterial tube having an interior opening therethrough, the arterial tube having a distal end configured for attachment to an artery of a human so that the interior of the arterial tube is capable of communicating with an interior of the artery, and at least two venous tubes each having a proximal end interconnected with the arterial tube and a distal end capable of being attached to a vein of the human with each of the at least two venous tubes connected to the vein at a different location, each venous tube having an interior opening therethrough with the venous tube openings each communicating between the arterial tube interior opening and an interior of the vein of the human so that blood from the artery of the human is capable of flowing into the arterial tube and out of the venous tubes into the vein of the human;
a means for interconnecting a kidney dialysis machine to the tubular configuration so that the blood flows through the kidney dialysis machine prior to flowing into the vein of the human.

2. The graft of claim 1 wherein the tubular configuration is in the shape of a Y with the arterial tube comprising the leg of the Y and the two venous tubes comprising the arms of the Y.

3. The graft of claim 1 wherein the distal end of the arterial tube and the distal ends of the venous tubes are each capable of attachment to the artery and the vein respectively by a sewing means.

4. The graft of claim 3 wherein each of the distal ends is capable of being sealed to the artery and the vein with a sealant.

5. The graft of claim 3 wherein the tubular configuration is fabricated of PTFE.

6. The graft of claim 3 wherein the tubular configuration is fabricated of Dacron.

7. The graft of claim 1 wherein each of the tubes has a diameter of at least 6 mm.
